(19) United States
(12) Patent Application Publication
(10) Pub. No.: US 2026/0007347 A1
(43) Pub. Date: Jan. 8, 2026

(54) LINEAR FLEXIBLE ELECTRODE FOR PERIPHERAL NERVE AND MANUFACTURING METHOD THEREOF

(71) Applicant: CENTER FOR EXCELLENCE IN BRAIN SCIENCE AND INTELLIGENCE TECHNOLOGY, CHINESE ACADEMY OF SCIENCES, Shanghai (CN)

(72) Inventors: Xue LI, SHANGHAI (CN); Zhengtuo ZHAO, SHANGHAI (CN); Xiaocheng LI, SHANGHAI (CN)

(21) Appl. No.: 18/875,680

(22) PCT Filed: Jun. 29, 2022
(86) PCT No.: PCT/CN2022/102315

§ 371 Date: Dec. 16, 2024

(30) Foreign Application Priority Data
Jun. 17, 2022 (CN) 202210688548.4

(51) Int. Cl. A61B 5/279; A61B 5/00; A61B 5/251; A61B 5/262; A61B 5/268; A61N 1/05

(52) U.S. Cl. CPC A61B 5/279; A61B 5/251; A61B 5/262; A61B 5/268; A61B 5/6877; A61B 5/6883; A61N 1/0558; A61B 2562/0209; A61B 2562/125; A61B 2562/16; A61B 2562/227

(57) ABSTRACT

The present disclosure relates to a linear flexible electrode for a peripheral nerve and a manufacturing method thereof. A linear flexible electrode for a peripheral nerve is provided, wherein the flexible electrode includes an implantation portion and a fixing portion, wherein at least part of the implantation portion is implantable into a peripheral nerve bundle, and the fixing portion is configured to fix the flexible electrode to the peripheral nerve bundle or other tissues in the vicinity of the peripheral nerve bundle, wherein: the flexible electrode includes a first insulation layer, a second insulation layer and a wire layer between the first insulation layer and the second insulation layer; and the implantation portion includes one or more electrode sites, each electrode site is electrically coupled to one of the wires in the wire layer, and in contact with the peripheral nerve after the flexible electrode is implanted into the peripheral nerve bundle to collect electrical signals from the peripheral nerve and transmit the collected electrical signals through the wires, or apply received electrical signals through the wires to the peripheral nerve.

### CROSS-REFERENCE TO RELATED APPLICATIONS

The present application is based on and claims priority to China Patent Application No. 202210688548.4 filed on Jun. 17, 2022, the disclosure of which is incorporated by reference herein in its entirety.

### TECHNICAL FIELD

The present disclosure relates to the field of life science technology, and more particularly relates to a linear flexible electrode for a peripheral nerve and a manufacturing method thereof.

### BACKGROUND

Peripheral Nervous System (PNS) which originates from the central nervous system and leads to all parts of human body, may be divided into somatic nervous system and autonomic nervous system. The peripheral nervous system is responsible for communicating with all parts of the body and plays the role of afferent and efferent information. Studies have shown that electrical stimulation may promote axonal regeneration and functional rehabilitation of peripheral nerve injury, and may also relieve neuropathic pains. Peripheral Nerve Stimulation (PNS) may apply low-level electrical pulses to painful nerves, so as to generate algesia interference. The stimulation of these non-painful sensory pathways seems to be attenuatedly input from nearby pain pathways. PNS may be applied together with superficial electrodes, for example, c nerve to treat leg pains. As another alternative, the subcutaneous electrode may be placed in a general area of pains, for example, waist pains. PNS has been applied to many pain symptoms, including complex area pain syndrome, diabetic peripheral neuropathy, ilio-inguinal neuralgia, intercostal neuralgia, lateral femoral cutaneous neuropathy (allodynia), lumbodynia, cervicodynia, nerve injury, peripheral neuropathy, post-amputation stump pain, postherpetic neuralgia, post-operative hernia pain, post-thoracotomy syndrome, trigeminal neuralgia, and other types of pains.

At present, common nerve electrodes include extrafascicular nerve electrodes implanted laterally of a nerve bundle and intrafascicular nerve electrodes implanted medially of a nerve bundle. The extrafascicular nerve electrodes are wrapped laterally of peripheral nerve bundle when applied. Common extrafascicular electrodes, such as Cuff electrode and FINE electrode, are assembled on the inner wall of a silicone hose or a flat catheter during the use, and then the silicone hose or the flat catheter is wrapped on the outer side of the nerve bundle.

Linear intrafascicular electrodes arrange electrode recording ends on an elongated substrate and insert the substrate into the nerve bundle when applied.

### SUMMARY

In the following, a brief summary of the present disclosure is given in order to provide a basic understanding of some aspects of the present disclosure. However, it should be understood that, this summary is not an exhaustive summary of the present disclosure. It is not intended to determine key parts or important parts of the present disclosure, nor is it intended to define the scope of the present disclosure. Its purpose is only to present some concepts of the present disclosure in a simplified form as a prelude to a more detailed description made hereinafter.

According to a first aspect of the present disclosure, a linear flexible electrode for a peripheral nerve is provided, the flexible electrode comprising an implantation portion and a fixing portion, wherein at least part of the implantation portion is implantable into a peripheral nerve bundle, and the fixing portion is configured to fix the flexible electrode to the peripheral nerve bundle or other tissues in the vicinity of the peripheral nerve bundle, wherein: the flexible electrode includes a first insulation layer, a second insulation layer and a wire layer between the first insulation layer and the second insulation layer; and the implantation portion includes one or more electrode sites, each electrode site is electrically coupled to one of the wires in the wire layer, and in contact with the peripheral nerve after the flexible electrode is implanted into the peripheral nerve bundle to collect electrical signals from the peripheral nerve and transmit the collected electrical signals through the wires, or apply received electrical signals through the wires to the peripheral nerve.

According to a second aspect of the present disclosure, a manufacturing method of a linear flexible electrode for a peripheral nerve is provided. The linear flexible electrode is the flexible electrode according to the first aspect of the present disclosure, and the method includes: manufacturing a first insulation layer, a wire layer, a second insulation layer and an electrode site on a substrate; and separating the flexible electrode from the substrate; wherein a via hole is manufactured at a position corresponding to the electrode site in at least one layer of the first insulation layer and the second insulation layer by patterning.

Other features and advantages of the present disclosure will become more explicit from the following detailed description of exemplary embodiments of the present disclosure with reference to the accompanying drawings.

### BRIEF DESCRIPTION OF THE DRAWINGS

The accompanying drawings which constitute part of this specification, describe the embodiments of the present disclosure, and together with this specification, serve to explain the principles of the present disclosure.

The present disclosure may be more explicitly understood from the following detailed description with reference to the accompanying drawings, in which:

FIG. 1 shows a schematic view of at least part of a linear flexible electrode for a peripheral nerve according to an embodiment of the present disclosure;

FIG. 2 shows a schematic view of an implantation position of a linear flexible electrode for a peripheral nerve according to an embodiment of the present disclosure;

FIG. 3 shows an exploded view of at least part of a linear flexible electrode for a peripheral nerve according to an embodiment of the present disclosure;

FIG. 4 shows a flowchart of a manufacturing method of a linear flexible electrode for a peripheral nerve according to an embodiment of the present disclosure;

FIG. 5 shows a schematic view of a manufacturing method of a linear flexible electrode for a peripheral nerve according to an embodiment of the present disclosure.

### DETAILED DESCRIPTION

The following detailed description is made with reference to the accompanying drawings and the following detailed description is provided to help fully understand various example embodiments of the present disclosure. The following description includes various details to help the understanding. However, these details are considered as examples only and not for the purpose of limiting the present disclosure, which is defined by the appended claims and their equivalents. The words and phrases used in the following description are only used for enabling to clearly and consistently understand the present disclosure. In addition, descriptions of well-known structures, functions and configurations might be omitted for the sake of clarity and conciseness. Those of ordinary skill in the art will appreciate that various changes and modifications may be made to the examples described herein without departing from the spirit and scope of the present disclosure.

The following descriptions of at least one exemplary embodiment which are in fact merely illustrative, shall by no means serve as any delimitation on the present disclosure as well as its application or use. In other words, the structures and methods herein are shown in an exemplary manner to illustrate different embodiments of the structures and methods in the present disclosure. However, those skilled in the art will understand that they merely illustrate exemplary methods of the present application that may be practiced, but not in an exhaustive way. Furthermore, the accompanying drawings are not necessarily drawn to scale, and some features might be exaggerated to show details of specific components.

The techniques, methods, and apparatuses known to those of ordinary skill in the relevant art might not be discussed in detail. However, the techniques, methods, and apparatuses shall be considered as a part of the granted description where appropriate.

Among all the examples shown and discussed here, any specific value shall be construed as being merely exemplary, rather than as being restrictive. Thus, other examples in the exemplary embodiments may have different values.

During the use, the silicone hose of the extrafascicular nerve electrode wrapped on the outer side of the nerve might be displaced relative to the nerve bundle. On the other hand, the signal sensed by the extrafascicular nerve electrode has a low resolution due to its implantation position. During the process of long use, the friction between the hard catheter of the extrafascicular nerve electrode and the nerve bundle may cause nerve damage, and the cicatrix thus formed on the nerve may affect the signal sensing and stimulation. In terms of the sensing signal, the extrafascicular nerve electrode can only sense a compound action potential signal or perform a wide range of nerve stimulation, and the sensing and stimulation accuracy is far from meeting the requirements of fine control.

The intrafascicular nerve interface may construct an interaction window between the peripheral nerve and the external through electrodes. On the one hand, the electrode may collect nerve information (for example, electrical signals from the peripheral nerves) for controlling external devices. On the other hand, it is possible to feed back external information to the peripheral nerve through stimulation (for example, electrical stimulation to be applied to peripheral nerves). At present, the intrafascicular nerve interface technology is increasingly regarded as an auxiliary technology, which helps to restore or enhance the cognitive and sensorimotor functions damaged by diseases or trauma, especially favorable for the rehabilitation of people with severe neuromuscular disabilities.

The existing hard intrafascicular nerve electrodes may cause severe long-term immune reaction after implantation since the mechanical properties between hard electrodes and soft nerve tissues are not matched during the use, which results in that the electrodes cannot conduct long-term stable signal sensing and stimulation.

The present disclosure provides a flexible electrode for electrophysiological signal sensing and stimulation of the peripheral nervous system. The flexible electrode which has a linear structure, may be used for acquiring electrical signals and applying functional electrical stimulation to the peripheral nerve after being implanted in the nerve bundle. By reducing the thickness of the electrode, it is possible to reduce the flex stiffness of the electrode, thereby solving the problem that the mechanical properties between the electrode and the peripheral nerve tissue are not matched and providing a long-term stable electrical signal sensing and stimulation interface. Moreover, the electrode array may be suitable for different human bodies or other vertebrates by proportionally increasing or decreasing the size of the electrode plate. Compared with the extrafascicular nerve electrode which can only record local field potential signals, the flexible electrode of the present disclosure may record an action potential and local field potential signals at the same time. Electrodes with different numbers of layers, different numbers of contacts, different sizes and shapes and contact distributions may be designed according to different needs, which is of great significance in the research of neuroscience and the application of rehabilitation medicine.

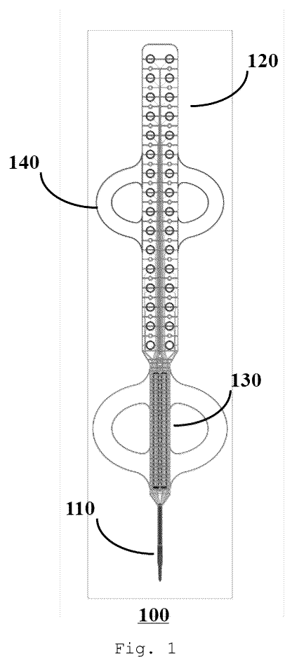

FIG. 1 shows an exploded view of at least part of a linear flexible electrode 100 for a peripheral nerve according to an embodiment of the present disclosure. As shown in FIG. 1, the flexible electrode 100 may include a linear implantation portion 110, wherein at least part of the linear implantation portion 110 may be implanted into the peripheral nerve bundle, so that it is possible to perform the sensing and stimulation of the motor neuron signals in the bundle of the peripheral nerve bundle as well as the deep and surface of the peripheral nerve bundle at the same time. Specifically, the implantation portion 110 may be implanted perpendicular to the nerve bundle, parallel to the nerve bundle, or at other angles with the nerve bundle. In the embodiment according to the present disclosure, the flexible electrode 100 may further include a back end portion 120, which may be implanted hypodermically and used for joining the flexible electrode 100 and a back end circuit for back end adaptation. The implantation portion 110 extends from the back end portion 120, and the flexible electrode 100 may further include a connection portion 130 between the implantation portion 110 and the back end portion 120.

After implantation, the flexible electrode for a peripheral nerve is in a long-term moving and changing in-vivo environment. Limb movement, external force and the like might cause relative displacement between the flexible electrode and the nerve bundle. In the present disclosure, the flexible electrode cannot generate a force against relative displacement through own deformation due to its own flexibility. In order to resist this relative displacement and ensure that the flexible electrode can accurately sense the electrical signal at the same position of the peripheral nerve and apply electrical stimulation at the same position of the peripheral nerve after implantation, the flexible electrode 100 may further include a fixing portion 140, which may be configured to fix the flexible electrode 100 to the peripheral nerve bundle or other tissues in the vicinity of the peripheral nerve bundle (such as but not limited to muscles and bones in the vicinity of the peripheral nerve bundle). As shown in FIG. 1, the fixing portion 140 may include a pore in the flexible electrode 100, wherein the fixing device (such as a suture) may pass through the pore and is attached to the peripheral nerve bundle or other tissues in the vicinity of the peripheral nerve bundle so as to reliably fix the flexible electrode 100. In the embodiment according to the present disclosure, the fixing portion 140 may be thicker than other parts of the flexible electrode 100 (such as the implantation portion 110, the back end portion 120 and the connection portion 130), to provide higher mechanical strength. In the embodiment shown in FIG. 1, the flexible electrode 100 includes a pair of fixing portions 140 located on both sides of the back end portion 120 and a pair of fixing portions 140 located on both sides of the connection portion 130. However, it should be understood that, the flexible electrode according to the present disclosure is not limited thereto, but may include more or less fixing portions. For example, the flexible electrode 100 may not include a pair of fixing portions 140 located on both sides of the connection portion 130. For example, the flexible electrode 100 includes a plurality of pairs of fixing portions 140 located on both sides of the back end portion 120.

In the embodiment according to the present disclosure, the implantation portion 110 may further have a mounting hole 111 (not shown) through which the electrode implantation device may be attached to the flexible electrode 100, and drive the flexible electrode 100 through the mounting hole 111 during implantation, thereby guiding the flexible electrode 100 to complete the implantation process.

The flexible electrode 100 shown in FIG. 1 includes an implantation portion 110 with a slender needle shape, a linear back end portion 120 and connection portion 130, and a semi-annular fixing portion 140. However, it should be understood that FIG. 1 shows only a non-limiting example, and the flexible electrode for the peripheral nerve bundle may have the implantation portion 110, the back end portion 120, the connection portion 130 and the fixing portion 140 with different shapes and sizes as required.

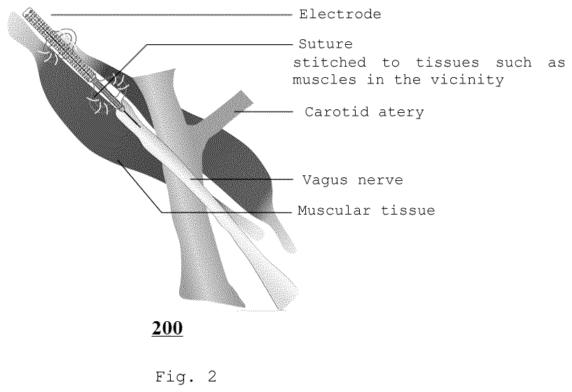

FIG. 2 shows a schematic view of an implantation position of a linear flexible electrode for the peripheral nerve according to an embodiment of the present disclosure. As shown in FIG. 2, at least part of the implantation portion of the flexible electrode is implanted into the vagus nerve, and the suture passing through the pore in the fixing portion of the flexible electrode is stitched to tissues such as muscles around the vagus nerve so as to fix the flexible electrode to the muscle tissue.

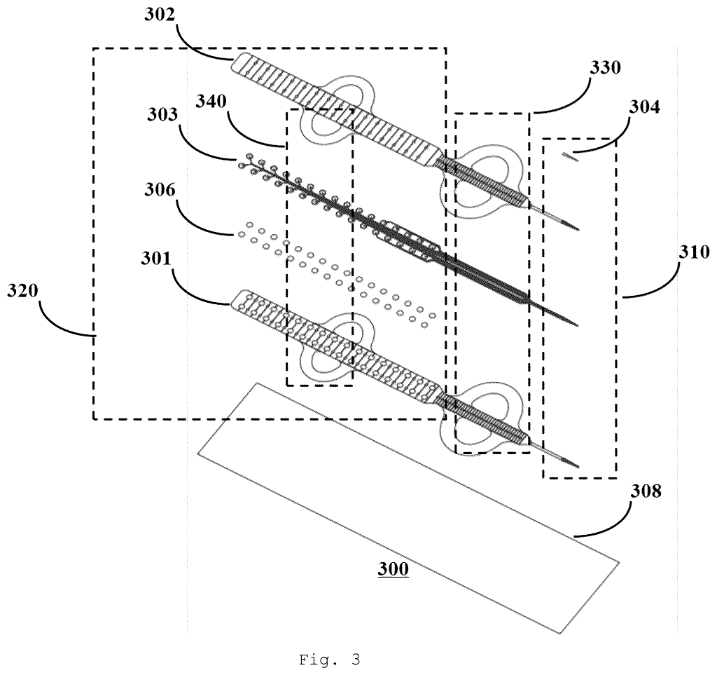

FIG. 3 shows an exploded view of at least part of a linear flexible electrode 300 for a peripheral nerve according to an embodiment of the present disclosure. As may be clearly seen from FIG. 3, the flexible electrode 300 has a multi-layer structure. Specifically, it includes a bottom insulation layer 301, a top insulation layer 302, a wire layer 303, an electrode site layer 304, a back end site layer 306, a flexible separation layer 308, and the like. It should be understood that, the layers of the flexible electrode 300 shown in FIG. 3 are only non-limiting examples, and the flexible electrode in the present disclosure may omit one or more layers therein. For example, it is possible to only include the bottom insulation layer, the top insulation layer and the wire layer therein, or it is also possible to include more other layers.

The flexible electrode 300 may include an insulation layer 301 located at the bottom and an insulation layer 302 located at the top. Specifically, as shown in FIG. 3, the implantation portion 310, the back end portion 320, the connection portion 330 and the fixing portion 340 of the flexible electrode 300 may each include insulation layers 301 and 302. The insulation layer in the flexible electrode may refer to the outer surface layer that produces an insulating effect in the electrode. Since the insulation layer of the flexible electrode needs to be in contact with the peripheral nerve after implantation, the material of the insulation layer is required to present a favorable insulation and a favorable biocompatibility at the same time. In the embodiment of the present disclosure, the materials of the insulation layers 301 and 302 may include Polyimide (PI), polydimethylsiloxane (PDMS), Parylene, epoxy resin, polyamideimide (PAI), SU-8 photoresist, silica gel, silicone rubber and the like. In the embodiment according to the present disclosure, in order to allow the flexible electrode to further present a biodegradable property, the materials of the insulation layers 301 and 302 may also include polylactic acid, polylactic acid-glycolic acid copolymer and the like. In addition, the insulation layers 301, 302 are also main parts that provide strength in the flexible electrode 300. If the insulation layer is too thin, it is possible to reduce the strength of the electrode, and if the insulation layer is too thick, it is possible to reduce the flexibility of the electrode. Moreover, the implantation of the electrode including an insulation layer that is too thick may bring great damage to the organism. In the embodiment according to the present disclosure, the thickness of the insulation layers 301, 302 may be 100 nm to 300 μm, preferably 300 nm to 20 μm.

The flexible electrode 300 may further include one or more wires spaced apart from each other in the wire layer 303 between the bottom insulation layer 301 and the top insulation layer 302. Specifically, as shown in FIG. 3, the implantation portion 310, the back end portion 320 and the connection portion 330 of the flexible electrode 300 may each include a wire layer 303. In the embodiment according to the present disclosure, each wire in the wire layer 303 may extend to the implantation portion 310 along the back end portion 320 and the connection portion 330, and extend to an electrode site located in the implantation portion 310 in the implantation portion 310. In the embodiment according to the present disclosure, the flexible electrode 300 may include one or more wires in the same wire layer 303, wherein each wire may be electrically coupled to an electrode site in the electrode site layer 304 and electrically coupled to a back end site in the back end site layer 306. In the embodiment according to the present disclosure, the thickness of the wire layer 303 and each conductor therein may be 5 nm to 200 μm. The distance between the wires may be as low as 10 nm, for example. The line width of the wires and the distance between the wires may be, for example, 10 nm to 500 μm, for example, 300 nm to 3 μm. It should be understood that, the dimensions and the like of the wires are not limited to the ranges listed above, but may be changed according to design requirements.

In the embodiment according to the present disclosure, the wires in the wire layer 303 may be a film structure including a plurality of sub-layers in the thickness direction. The material of these sub-layers may be materials that may enhance the adhesion, ductility, conductivity and the like of the wires. As a non-limiting example, the wire layer 303 may be a metal film including three sub-layers, wherein the first sub-layer and the second sub-layer in contact with the insulation layers and 302 respectively are adhesive sub-layers, which may use metal adhesive materials such as titanium (Ti), titanium nitride (TiN), chromium (Cr), tantalum (Ta) or tantalum nitride (TaN) or nonmetallic adhesive materials. The third sub-layer located between the first sub-layer and the second sub-layer is a conductive sub-layer, which may use materials with a favorable conductivity such as gold (Au), platinum (Pt), iridium (Ir), tungsten (W), platinum-iridium alloy, titanium alloy, graphite, carbon nanotubes and PEDOT. In the embodiment according to the present disclosure, in order to allow the flexible electrode to further present a biodegradable property, the conductive sub-layer may also use materials such as magnesium (Mg), molybdenum (Mo) and their alloys. It should be understood that, the wire layer may be made of other metallic materials or nonmetallic materials with conductivity, and may also be made of polymer conductive materials and composite conductive materials. In the embodiment according to the present disclosure, the thickness of the adhesion sub-layer may be 1 nm to 50 nm.

The flexible electrode 300 may further include one or more electrode sites in the top electrode site layer 304, and each electrode site is electrically coupled to one of the wires in the wire layer 303, and in contact with the peripheral nerve after implantation of the flexible electrode 300 to collect electrical signals from the peripheral nerve and transmit the collected electrical signals through the wires, or apply the received electrical signals through the wires to the peripheral nerve. In the flexible electrode 300 shown in FIG. 3, the electrode sites in the implantation portion 110 located on the surface of the peripheral nerve after implantation may be used to apply or collect electrical signals on the surface of the peripheral nerve, and the electrode sites located inside the peripheral after implantation may be used to apply or collect electrical signals inside the peripheral nerve. In addition, since each electrode site is coupled to its corresponding wire, when the flexible electrode 300 is used as a stimulating electrode, each electrode site therein may synchronously or asynchronously apply the same or different electrical signals to a deep portion of the surface of the peripheral nerve and/or different positions on the surface. Moreover, when the flexible electrode 300 is used as a sensing electrode, these electrode sites may simultaneously and finely collect the electrical signals at a deep portion of the surface of the peripheral nerve and/or different positions on the surface.

The electrode site in the top electrode site layer 304 may be electrically coupled to a corresponding wire in the wire layer through a via hole in the top insulation layer 302 at a position corresponding to the electrode site. In the case where the flexible electrode includes a plurality of wires, the flexible electrode may correspondingly include a plurality of electrode sites in the top electrode site layer 304, and these electrode sites are each electrically coupled with one of the plurality of wires through a corresponding via hole in the top insulation layer 302. In the embodiment according to the present disclosure, the electrode site in the top electrode site layer 304 may be a film structure including a plurality of sub-layers in the thickness direction. The material of the adhesion sub-layer close to the wire layer 303 among the plurality of sub-layers may be a material that may enhance the adhesion between the electrode site and the wire, and the thickness of the adhesion sub-layer may be 1 nm to 50 nm. As a non-limiting example, the electrode site layer 304 may be a metal film including two sub-layers, wherein the first sub-layer close to the wire layer 303 is Ti, TiN, Cr, Ta or TaN, and the second sub-layer of the electrode site layer 304 exposed to the outside is Au. It should be understood that, the electrode site layer may also be similar to the wire layer, and made of other metallic materials or nonmetallic materials with conductivity, such as Pt, Ir, W, Mg, Mo, platinum-iridium alloy, titanium alloy, graphite, carbon nanotubes and PEDOT.

Each electrode site may have a micron-scale plane size and a nanometer-scale thickness. In the embodiment according to the present disclosure, the shapes of the electrode sites may be set to be various regular or irregular shapes as required. The number may be one or more, the maximum side length or diameter may be 1 μm to 500 μm, the distance between the electrode sites may be 1 μm to 5 mm, and the thickness may be 5 nm to 200 μm. It should be understood that, the shape, number, size, distance and the like of the electrode sites may be selected according to the condition of a peripheral nerve area to be sensed or stimulated.

In the embodiment according to the present disclosure, the surface of the electrode site in contact with the peripheral nerve tissue exposed to the outside may also have a surface modification layer to improve the electrochemical properties of the electrode site. As a non-limiting example, the surface modification layer may be obtained by a method such h as electrically initiated polymerization coating and sputtering iridium oxide film using PEDOT: PSS, to reduce the impedance (such as electrochemical impedance at 1 kHz working frequency) in the case where the flexible electrode collects electrical signals, and to improve the charge injectivity in the case where the flexible electrode is stimulated by applying electrical signals, so as to improve the interaction efficiency.

In the embodiment shown in FIG. 3, the top electrode site layer 304 is located on the top insulation layer 302. It should be understood that, the electrode site layer 304 may be located outside at least one of the bottom insulation layer 301 and the top insulation layer 302, so that the electrode site is located on the outer surface of the flexible electrode 300 to facilitate contact with the nerve bundle. For example, in the embodiment according to the present disclosure, the flexible electrode may further include an electrode site located in the bottom electrode site layer 305 under the bottom insulation layer 301, and the electrode site in the bottom electrode site layer 305 after the flexible electrode is implanted may be in contact with peripheral nerve tissue to directly collect or apply electrical signals. Similar to the electrode site in the top electrode site layer 304, in the flexible electrode 300, the electrode site in the bottom electrode site layer 305 may be electrically coupled to a corresponding wire in the wire layer through a via hole in the bottom insulation layer 301 at a position corresponding to the electrode site. In the embodiment according to the present disclosure, the electrode site in the bottom electrode site layer 305 and the electrode site in the top electrode site layer 304 may be located at opposite positions at the top and bottom of the flexible electrode 300, and the electrode site in the bottom electrode site layer 305 and the electrode site in the top electrode site layer 304 located at an opposite position are electrically coupled to the same wire in the wire layer 303. In the embodiment according to the present disclosure, the electrode site in the bottom electrode site layer 305 and the electrode site in the top electrode site layer 304 may also be located at different positions at the top and bottom of the flexible electrode 300, so as to collect or apply electrical signals in different areas of the peripheral nerve. Moreover, in the embodiment according to the present disclosure, the electrode site in the bottom electrode site layer 305 may also be electrically coupled to different wires in the wire layer 303 from that in the top electrode site layer 304.

It should be understood that, the bottom electrode site layer 305 is an optional but not necessary part of the flexible electrode. For example, the flexible electrode in the present disclosure may include only the top electrode site layer 304 but not the bottom electrode site layer 305. The shape, size, material and the like of the bottom electrode site may be similar to the top electrode site, which will not be described in detail here.

By including an electrode site layer different from the wire layer 303 in the flexible electrode 300, that is, separating the electrode site in the flexible electrode 300 and the wire in different layers, it is possible to advantageously reduce the size of the electrode, and provide an electrode with higher integration. However, in the embodiment according to the present disclosure, it is also possible to not include a separate electrode site layer. In this case, the electrode site of the flexible electrode and the wire may be both located in the wire layer, and the electrode site is exposed to the outer surface of the flexible electrode through a via hole in at least one of the bottom insulation layer and the top insulation layer, so that the electrode site may be in direct contact with the peripheral nerve tissue.

In the embodiment of the present disclosure, the flexible electrode may further include an additional wire layer, that is, the flexible electrode in the present disclosure may include one or more wire layers. The size, material, manufacturing method and the like of the additional wire layer may be similar to the wire layer 303, which will not be described in detail here. In the case where the flexible electrode includes an additional wire layer, these wire layers may be spaced apart by additional insulation layers, and each wire layer includes a plurality of wires spaced apart from each other. The size, material, manufacturing method and the like of the additional insulation layer may be similar to the bottom insulation layer 301 and/or the top insulation layer 302, which will not be described in detail here. One or more wires in these additional wire layers may be electrically coupled to the electrode sites located below the bottom insulation layer or on the top insulation layer through a via hole in one or more of the bottom insulation layer, the top insulation layer and the additional insulation layer. By including a plurality of wire layers in the flexible electrode, it is possible to improve the number and accuracy of signals transmitted through the flexible electrode in the case of the same cross-sectional width, that is, providing a multi-channel electrode with high accuracy, and favorable for realizing high-flux interaction.

In the embodiment according to the present disclosure, the back end portion 320 of the flexible electrode 300 may include a back end site in the back end site layer 306. The back end site may be electrically coupled to one of the wires (FIG. 3 shows that each back end site in the back end site layer 306 is electrically coupled to a metal ring at an extremity of one of the wires in the wire layer 303) and exposed to the outer surface of the flexible electrode through a via hole in at least one of the bottom insulation layer 301 and the top insulation layer 302. In this way, when the back end portion is connected to the back end circuit, it is possible to realize bidirectional signal transmission between the electrode site electrically coupled to the wire and the back end circuit. In the embodiment according to the present disclosure, the back end site may be located between at least one of the top insulation layer and the bottom insulation layer and the wire layer, and exposed through a via hole in the other of the top insulation layer and the bottom insulation layer. For example, as shown in FIG. 3, the back end site is located between the wire layer 303 and the bottom insulation layer 301, and the back end site may be electrically coupled to the back end circuit through the metal ring of the wire layer 303 and the via hole in the top insulation layer 302. In this arrangement, the back end site is not placed on the outer surface of the flexible electrode 300, which is favorable for a stable connection between the flexible electrode 300 and the back end circuit. Here, the back end circuit may refer to a circuit at the back end of the flexible electrode, such as a signal recording circuit, a signal processing circuit and a signal generating circuit associated with the signal of the flexible electrode. In the embodiment according to the present disclosure, the flexible electrode may be coupled to the back end circuit in a connection method. Specifically, the Ball Gate Array (BGA) package site as the back end site may be transferred to the commercial signal recording system through a Printed Circuit Board (PCB), a Flexible Printed Circuit board (FPC) and the like, and the connection method includes ball mounting and anisotropic conductive film bonding (ACF Bonding). After the flexible electrode is manufactured, the flexible electrode may be separated from the substrate manufactured therefrom (for example, separated from the substrate by removing its flexible separation layer), and the flexible electrode may be then connected (for example, soldered) to the back end circuit. In the embodiment according to the present disclosure, the flexible electrode and the back end circuit connected to the back end portion may be encapsulated together by any of epoxy resin and polydimethylsiloxane or a combination thereof to improve their strength. In the embodiment according to the present disclosure, the encapsulating gap between the back end portion and the back end circuit is coated with high-viscosity waterproof glue to ensure the waterproof property of the connection between the flexible electrode and the back end circuit after implantation.

The back end site may have a micron-scale plane size and a nanometer-scale thickness. As a non-limiting example, the back end site may be a BGA encapsulating site with a diameter of 50 μm to 2000 μm, or a elliptical, circular, rectangular, rounded rectangular or chamfered rectangular site with a side length of 50 μm to 2000 μm. The thickness of the back end site layer 306 and the back end site therein may be 5 nm to 200 μm. It should be understood that, the shape, size and the like of the back end site are not limited to the range listed above, but may be changed according to design needs.

The back end site in a connection method may include a plurality of sub-layers in the thickness direction. The material of the adhesion sub-layer close to the wire layer 303 among the plurality of sub-layers may be a material that may enhance the adhesion between the electrode site and the wire, and the material of the intermediate soldering flux sub-layer among the plurality of sub-layers may be a soldering flux material. The conductive sub-layer among the plurality of sub-layers may use other metallic materials or nonmetallic materials with conductivity of the wire layer as described previously, and the outermost layer among the plurality of sub-layers that r might be exposed through the insulation layers 301 and 302 is an anti-oxidation protective sub-layer. As a non-limiting example, the back end site layer 306 may be a metal film including three sub-layers, wherein the first layer close to the wire layer 303 may be an adhesion sub-layer of nanometer order to improve the adhesion between the back end site layer 306 and the wire layer 303. The second layer as a soldering flux sub-layer may be nickel (Ni), Pt or palladium (Pd), and the third layer as a conductive sub-layer may be Au, Pt, Ir, W, Mg, Mo, platinum-iridium alloy, titanium alloy, graphite, carbon nanotubes, PEDOT and the like. It should be understood that, the back end site layer may also be made of other metal materials or non-metal materials with conductivity. The back end site layer 306 in FIG. 3 is a part connected with the back end processing system or chip, and the size, distance, shape and the like of its site may be changed in design according to different connection methods of the back end.

In the embodiment according to the present disclosure, the flexible electrode 300 and the back end circuit connected to the back end portion 320 may be encapsulated together by any of epoxy resin and polydimethylsiloxane and a combination thereof to improve their strength. After encapsulation, the flexible electrode 300 may be immersed in a biocompatible adhesive (for example but not limited to low-modulus silicone), which produces a waterproof effect. After the flexible electrode 300 is coated with a biocompatible adhesive, the flexible electrode 300 may be implanted.

In the embodiment according to the present disclosure, the flexible electrode may not include a back end site layer. In this case, the back end site for adaptation in the back end portion of the flexible electrode may be a part in the wire layer and electrically coupled to a corresponding wire in the wire layer, and exposed to the outer surface of the electrode through a corresponding via hole in at least one of the top insulation layer and the bottom insulation layer.

In the embodiment according to the present disclosure, the flexible electrode 300 may further include a flexible separation layer 308. The flexible separation layer 308 of the flexible electrode 300 in FIG. 3 is shown to be located at the lowermost layer of the entire flexible electrode. That is, it should be understood that, the position of the flexible separation layer is not limited thereto, and the flexible electrode may include one or more flexible separation layers located at different positions. Preferably, the flexible separation layer may be manufactured between the substrate and the bottom insulation layer. The flexible separation layer may use a material that may be removed by a specific substance (for example, a solution) to separate two parts of the flexible electrode above and below the flexible separation layer, and avoid damage to the flexible electrode at the same time. Specifically, the flexible separation layer may be used to separate the whole electrode or only a flexible portion of the electrode from the substrate, separate the flexible substrate from the hard substrate, and separate a portion with excessive adhesion and required to be separated. In the embodiment of the present disclosure, the material of the flexible separation layer may be metal or nonmetal materials such as Ni, Cr and aluminum (Al).

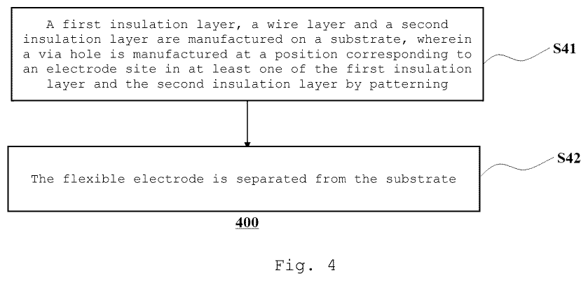

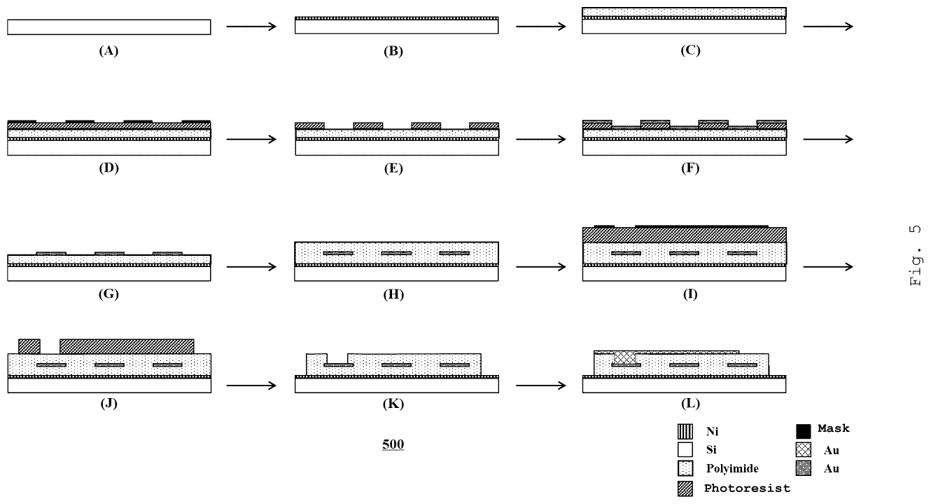

FIG. 4 shows a flowchart of a method 800 of manufacturing a flexible electrode according to an embodiment of the present disclosure. In the present disclosure, a manufacturing method based on micro-electro-mechanical system (MEMS) process may be used to manufacture a nano-scale flexible electrode. As shown in FIG. 4, the method 400 may include: in S41, a first insulation layer, a wire layer and a second insulation layer are manufactured on a substrate, wherein a via hole is manufactured at a position corresponding to an electrode site in at least one of the first insulation layer and the second insulation layer by patterning; and in S42, the flexible electrode is separated from the substrate. The step of manufacturing each layer of the flexible electrode in S41 will be described in detail below in conjunction with FIG. 5.

FIG. 5 shows a schematic view of a method 500 of manufacturing a flexible electrode according to an embodiment of the present disclosure. The manufacturing process and structure of the parts of the flexible electrode such as a flexible separation layer, a bottom insulation layer, a wire layer, a top insulation layer and an electrode site layer will be described in more detail in conjunction with FIG. 5.

The view (A) of FIG. 5 shows the substrate of the electrode. In the embodiment according to the present disclosure, a hard substrate such as glass, quartz and silicon wafer may be used. In the embodiment of the present disclosure, other soft materials may also be used as the substrate, such as the same material as the insulation layer.

The view (B) of FIG. 5 shows a step of manufacturing a flexible separation layer on the substrate. The flexible separation layer may be removed by applying a specific substance, so as to facilitate separating the flexible portion of the electrode from the hard substrate. The embodiment shown in FIG. 5 uses Ni as the material of the flexible separation layer, and other materials such as Cr and Al may also be used. In the embodiment according to the present disclosure, when the flexible separation layer is manufactured on the substrate by evaporation, part of the exposed substrate may be first etched, so as to improve the flatness of the entire substrate after evaporation. It should be understood that, the flexible separation layer is an optional but not necessary part of the flexible electrode. According to the properties of the selected material, the flexible electrode may also be conveniently separated in the case where there is no flexible separation layer. In the embodiment according to the present disclosure, the flexible separation layer may also have a mark for the alignment of subsequent layers.

The view (C) of FIG. 5 shows that the insulation layer at the bottom is manufactured on the flexible separation layer. As a non-limiting example, in the case where the insulation layer is made from polyimide, the manufacturing of the insulation layer at the bottom may include the steps of film-forming process, film-forming curing and reinforced curing to manufacture a film as an insulation layer. The film forming process may include coating polyimide on the flexible separation layer, for example, a layer of polyimide may be spin-coated at a segmented rotational speed. Film-forming curing may include gradually heating to a high temperature and performing thermal insulation to form a film, so as to carry out subsequent machining steps. Reinforced curing may include multi-gradient heating, preferably in vacuum or nitrogen atmosphere, and performing baking for several hours before manufacturing subsequent layers. It should be understood that, the above-described manufacturing process is only a non-limiting example of the manufacturing process of the bottom insulation layer, and one or more steps therein may be omitted, or more other steps may be included.

It should be noted that, the above-described manufacturing process is directed to the embodiment in which the bottom insulation layer in the flexible electrode without the bottom electrode site layer is manufactured, and there is no via hole corresponding to the electrode site in the bottom insulation layer. If the flexible electrode includes a bottom electrode site layer, the bottom electrode site layer may be first manufactured on the flexible separation layer before the bottom insulation layer is manufactured. For example, Au and Ti may be sequentially evaporated on the flexible separation layer. The patterning step of the bottom electrode site will be described in detail concerning the top electrode site hereinafter. Accordingly, in the case where the flexible electrode includes a bottom electrode site, during the process of manufacturing the bottom insulation layer, in addition to the above-described steps, a patterning step may also be included for etching a via hole at a position corresponding to the bottom electrode site in the bottom insulation layer. The patterning step of the insulation layer will be described in detail concerning the top electrode site hereinafter.

The views (D) to (G) of FIG. 5 show that a wire layer is manufactured on the insulation layer at the bottom. As shown in view (D), a photoresist and a mask may be applied above the insulation layer at the bottom. It should be understood that, other lithography methods may also be used to prepare a patterned film, such as laser direct writing and electron beam lithography. In the embodiment according to the present disclosure, for a metal film such as a wire layer, a double-layer adhesive may be applied to facilitate the manufacture (evaporation or sputtering) and peeling of the patterned film. By setting a pattern of the mask related to the wire layer, for example, the pattern of the wire layer described previously, such as the pattern of the wire layer 303 in FIG. 3, may be realized. Next, exposure and development may be carried out to obtain the structure shown in the view (E). In the embodiment according to the present disclosure, the exposure may use contact lithography, and the mask and the structure are exposed in a vacuum contact mode. In the embodiment according to the present disclosure, different developing s and their concentrations may be used for different sizes of patterns. In the embodiment according to the present disclosure, this step may further include alignment between layers. Next, film forming may be performed on the structure shown in the view (E), for example, a process such as evaporation and sputtering may be used to deposit a metal film material, such as Au, so as to obtain the structure shown in the view (F). Next, peeling may be performed to separate the film of the non-patterned area from the film of the patterned area by removing photoresist in the non-patterned area, so as to obtain the structure shown in the view (G), that is, the wire layer is manufactured. In the embodiment according to the present disclosure, after stripping peeling, stripping treatment may be performed again to further remove the residual glue on the surface of the structure.

In the embodiment according to the present disclosure, the back end site layer may also be manufactured before the wire layer is manufactured. As a non-limiting example, the manufacturing process of the back end site layer may be similar to the manufacturing process of the metal film described previously with respect to the wire layer.

The views (H) to (K) of FIG. 5 show the manufacturing of the insulation layer at the top. For a photosensitive film, patterning may be generally realized directly by patterned exposure and development. For a non-photosensitive material used in the insulation layer, patterning may not be realized by performing exposure and development thereon. Therefore, a patterned etching-resistant layer that is thick enough may be manufactured on this layer, and then the film in the area not covered by the etching-resistant layer may be removed by dry etching (at the same time, the etching-resistant layer may also be thinned, so that it is necessary to ensure that the etching-resistant layer is thick enough), and the etching-resistant layer may be removed to realize the patterning of the non-photosensitive layer. As a non-limiting example, the manufacturing of the insulation layer may use a photoresist as an etching resistant layer. The manufacturing of the top insulation layer may include steps such as film-forming process, film-forming curing, patterning and reinforced curing, wherein the view (H) shows the structure obtained after film-forming of the top insulation layer, the view (I) shows the application of a photoresist and a mask on the top insulation layer after film-forming, the view (J) shows the structure including the etching-resistant layer obtained after exposure and development, and the view (K) shows the structure including the manufactured top insulation layer. Film-forming process, film-forming curing and reinforced curing have been described in detail concerning the bottom insulation layer previously, which will be omitted here for the sake of simplicity. The patterning step may be performed after film-forming curing or reinforced curing, and the insulation layer after reinforced curing has a stronger etching-resistance. Specifically, in the view (I), a layer of photoresist that is thick enough is manufactured on the insulation layer through steps such as spin coating and baking. By setting the pattern of the mask related to the top insulation layer, for example, the pattern of the top insulation layer 302 shown in FIG. 3, that is, the contours of the implantation portion 310, the back end portion 320, the connection portion 330 and the fixing portion 340 of the flexible electrode 300, and the contours of the via holes realized at positions corresponding to the electrode site and the back end site in the top insulation layer 302 may be realized. In the view (J), the pattern is transferred to the photoresist on the insulation layer by steps such as exposure and development to obtain an etching-resistant layer, wherein the part to be removed from the top insulation layer is exposed. The exposed part of the top insulation layer may be removed by oxygen plasma etching, and the remaining photoresist on the top insulation layer may be removed by developing solution or acetone after flood exposure, so as to obtain the structure shown in the view (K).

In the embodiment according to the present disclosure, the top insulation layer may be subjected to viscosifying treatment before manufacturing so as to improve the bonding force between the bottom insulation layer and the top insulation layer.

The view (L) of FIG. 5 shows the manufacturing of the top electrode site layer on the top insulation layer.

In the embodiment of the present disclosure, during the process of manufacturing the flexible electrode, in order to make the thickness of the fixing portion of the flexible electrode greater than that of other parts of the flexible electrode, the top insulation layer and/or the bottom insulation layer of the fixing portion may be made thicker than that of the top insulation layer and/or the bottom insulation layer of other parts of the flexible electrode. Specifically, for example, in the case where it is necessary to make the top insulation layer of the fixing portion thicker than the top insulation layer of other parts, before making the top electrode site layer (for example, after the view (K) and before the view (1) in FIG. 5), a part of the thickness of the insulation layers of other parts of the flexible electrode than the fixing portion may be removed. This may be achieved by the following steps (not shown): applying a layer of photoresist and a mask to protect the fixing portion on the top insulation layer of the structure shown in the view (K); transferring the pattern to the photoresist on the top insulation layer through steps such as exposure and development to obtain an etching-resistant layer, wherein the part to be removed from the top insulation layer is exposed; and removing the etching resistant layer and a part of the thickness of the exposed top insulation layer of other parts than the fixing portion by etching. Since this method needs to remove part of the top insulation layer, in order to ensure that the top insulation layer of the obtained flexible electrode ( [symbol] ) still has a normal thickness to ensure the strength of the flexible electrode, when the top insulation layer (for example, the view (H) in FIG. 5) is manufactured, the top insulation layer may be manufactured thicker than that in a conventional circumstance (for example, in the case where it is not necessary to manufacture the fixing portion to be thicker than other parts).

The present disclosure provides a linear flexible electrode for a peripheral nerve and a manufacturing method thereof. The flexible electrode uses a flexible material instead of a hard silicon electrode, uses high polymer as an insulation layer to wrap a conductive material, and reduces the thickness of the electrode to reduce its flex stiffness, thereby improving the problem that the mechanical properties between the electrode and the tissue are not matched, and finally providing a long-term stable electrical signal sensing and stimulation interface. The flexible electrode which uses a linear design, may be implanted in the peripheral nerve bundle and in direct contact with a single nerve axon, so that it has a much higher selectivity and channel number than the extrafascicular nerve electrode. This electrode which has the characteristics of less trauma, is suitable for use in some minimally invasive scenes to achieve favorable sensing and stimulation functions of peripheral nerve signals.

The words “front”, “rear”, “top”, “bottom”, “above” and “below” in the specification and claims, if any, are used for descriptive purposes but not necessarily for describing a constant relative position. It should be understood that, the words thus used are interchangeable under appropriate circumstances, so that the embodiments of the present disclosure described here can, for example, perform operations in other orientations than those shown or otherwise described here.

As used here, the word “exemplary” means “serving as an example, instance or illustration”, rather than as a “model” to be accurately reproduced. Any implementation exemplarily described here is not necessarily to be construed as preferable or advantageous over other implementations. Moreover, the present disclosure is not defined by any expressed or implied theory provided in the above-described technical field, background, summary or detailed description.

As used here, the word “substantially” means including any slight change caused by design or manufacturing defects, tolerances of devices or elements, environmental influences and/or other factors. The word “substantially” also allows for differences between perfect or ideal instances caused by parasitic effects, noise and other practical considerations that might be present in actual implementations. For reference purposes only, similar terms such as “first” and “second” may be used herein, and thus are not intended to be restrictive.

For example, the words “first”, “second” and other such numerical words involving the structures or elements do not imply an order or sequence unless specified otherwise in the context.

It should also be understood that, the words “comprising/including” when used herein indicate the presence of the features, wholes, steps, operations, units and/or components as set forth, but do not exclude the presence or addition of one or more other features, wholes, steps, operations, units and/or components and/or combinations thereof.

As used herein, the terms “and/or” include any and all combinations of one or more of the associated listed items. The terms used herein which are for the purpose of describing specific embodiments only, are not intended to limit the present disclosure. As used herein, the singular forms “a”, “an” and “the” are also intended to include plural forms unless clearly indicated otherwise in the context.

Those skilled in the art should realize that, the boundaries between the above-described operations are merely illustrative. Multiple operations may be combined into a single operation, which may be distributed among additional operations, and performed at least partially overlapping in time. Moreover, alternative embodiments may include multiple examples of specific operations, and the operation sequence may be changed in other various embodiments. However, other modifications, changes and substitutions are also possible. Therefore, this specification and the accompanying drawings should be regarded as illustrative rather than restrictive.

Although some specific embodiments of the present disclosure have been described in detail by way of examples, those skilled in the art should understand that the above examples are only for an illustrative purpose, rather than limiting the scope of the present disclosure. Various embodiments disclosed here may be arbitrarily combined without departing from the spirit and scope of the present disclosure. Those skilled in the art should also understand that, multiple modifications may be made to the embodiments without departing from the scope and spirit of the present disclosure. The scope of the present disclosure is defined by the appended claims.

1. A linear flexible electrode for a peripheral nerve, the flexible electrode comprising an implantation portion and a fixing portion, wherein at least part of the implantation portion is implantable into a peripheral nerve bundle, and the fixing portion is configured to fix the flexible electrode to the peripheral nerve bundle or other tissues in the vicinity of the peripheral nerve bundle, wherein:
the flexible electrode comprises a first insulation layer, a second insulation layer and a wire layer between the first insulation layer and the second insulation layer; and
the implantation portion comprises one or more electrode sites, each electrode site is electrically coupled to one of the wires in the wire layer, and in contact with the peripheral nerve after the flexible electrode is implanted into the peripheral nerve bundle to collect electrical signals from the peripheral nerve and transmit the collected electrical signals through the wires, or apply received electrical signals through the wires to the peripheral nerve.

2. The flexible electrode according to claim 1, wherein the flexible electrode comprises a plurality of wire layers spaced apart by an additional insulation layer, and each wire layer comprises a plurality of wires spaced apart from each other.

3. The flexible electrode according to claim 1, wherein the fixing portion comprises a pore in the flexible electrode, and a fixing device is capable of passing through the pore and being attached to the peripheral nerve bundle or the other tissues to fix the flexible electrode to the peripheral nerve bundle or the other tissues.

4. The flexible electrode according to claim 1, wherein the other tissues comprise muscles or bones in the vicinity of the peripheral nerve bundle.

5. The flexible electrode according to claim 1, wherein the fixing portion is thicker than other parts of the flexible electrode to provide higher mechanical strength.

6. The flexible electrode according to claim 1, wherein:
the electrode sites are located on an outer side of at least one layer of the first insulation layer and the second insulation layer, and electrically coupled to the wires in the wire layer through a via hole in the at least one layer.

7. The flexible electrode according to claim 6, wherein the electrode sites comprise a conductive sub-layer, and a material of the conductive sub-layer is any of gold, platinum, iridium, tungsten, magnesium, molybdenum, platinum-iridium alloy, titanium alloy, graphite, carbon nanotubes and PEDOT or a combination thereof.

8. The flexible electrode according to claim 7, wherein the electrode sites further comprise an adhesion sub-layer close to the wire layer, and the adhesion sub-layer is made of a material capable of enhancing the adhesion between the electrode site and the wire layer.

9. The flexible electrode according to claim 1, wherein:
the electrode sites are located in the wire layer and exposed through a via hole in at least one layer of the first insulation layer and the second insulation layer.

10. The flexible electrode according to claim 1, wherein the electrode site is shaped as required, a number of the electrode sites is one or more, a maximum side length or diameter of the electrode site is 1 micron to 500 microns, a spacing between the electrode sites is 1 microns to 5 millimeters, and a thickness of the electrode site is 5 nanometers to 200 microns.

11. The flexible electrode according to claim 1, further comprising a back end portion implanted hypodermically and fixed by the fixing portion, wherein:
the back end portion comprises a back end site coupled to both one of the wires in the wire layer and a back end circuit to realize bidirectional signal transmission between the electrode sites electrically coupled to one of the wires and the back end circuit.

12. The flexible electrode according to claim 11, wherein the flexible electrode comprises a pair of fixing portions located on both sides of the back end portion.

13. The flexible electrode according to claim 12, wherein the flexible electrode further comprises a pair of fixing portions located on both sides of a connection portion of the flexible electrode, wherein the connection portion is a part of the flexible electrode located between the implantation portion and the back end portion.

14. The flexible electrode according to claim 11, wherein the back end site is located in the wire layer and exposed through a via hole in at least one layer of the first insulation layer and the second insulation layer.

15. The flexible electrode according to claim 11, wherein the back end site is located between the wire layer and at least one layer of the first insulation layer and the second insulation layer, and exposed through a via hole in the other layer of the first insulation layer and the second insulation layer.

16. The flexible electrode according to claim 15, wherein the back end site comprise a conductive sub-layer, and a material of the conductive sub-layer is any of gold, platinum, iridium, tungsten, magnesium, molybdenum, platinum-iridium alloy, titanium alloy, graphite, carbon nanotubes and PEDOT or a combination thereof.

17. The flexible electrode according to claim 15, wherein the back end site has thickness of 5 nanometers to 200 micrometers.

18. The flexible electrode according to claim 15, wherein the back end site further comprises an adhesion sub-layer close to the wire layer, and a material of the adhesion sub-layer is any of chromium, tantalum, tantalum nitride, titanium and titanium nitride or a combination thereof.

19. The flexible electrode according to claim 11, wherein the flexible electrode and the back end circuit connected to the back end portion are encapsulated together by any of epoxy resin and polydimethylsiloxane or a combination thereof.

20. The flexible electrode according to claim 19, wherein the flexible electrode is coated with a biocompatible adhesive after encapsulation.

21. The flexible electrode according to claim 1, wherein the wire layer comprises a conductive sub-layer, and a material of the conductive sub-layer is any of gold, platinum, iridium, tungsten, platinum-iridium alloy, titanium alloy, graphite, carbon nanotubes and PEDOT or a combination thereof.

22. The flexible electrode according to claim 21, wherein the conductive sub-layer has a thickness of 5 nanometers to 200 micrometers.

23. The flexible electrode according to claim 15, wherein the wire layer comprises a conductive sub-layer and an adhesion sub-layer close to any of the electrode site and the back end site, and the material of the adhesion sub-layer is any of chromium, tantalum, tantalum nitride, titanium and titanium nitride or a combination thereof.

24. The flexible electrode according to claim 1, wherein the first insulation layer and the second insulation layer have a thickness of 100 nanometers to 300 micrometers.

25. The flexible electrode according to claim 1, wherein a material of the first insulation layer and the second insulation layer are any of polyimide, polydimethylsiloxane, parylene, epoxy resin, polyamideimide, SU-8 photoresist, silica gel and silicone rubber or a combination thereof.

26. The flexible electrode according to claim 1, further comprising a flexible separation layer, wherein the flexible separation layer is removable by a specific substance to separate a part of the flexible electrode and avoid damage to the flexible electrode.

27. The flexible electrode according to claim 26, wherein a material of the flexible separation layer is any of nickel, chromium and aluminum or a combination thereof.

28. The flexible electrode according to claim 26, wherein the flexible separation layer further comprises an adhesion sub-layer, and a material of the adhesion sub-layer is chromium, tantalum, tantalum nitride, titanium or titanium nitride.

29. The flexible electrode according to claim 1, wherein the implantation portion comprises a mounting hole through which the electrode implantation device is attached to the flexible electrode for implantation.

30. The flexible electrode according to claim 1, wherein a material of the wire layer is any of magnesium, molybdenum and their alloys or a combination thereof, and a material of the first insulation layer and the second insulation layer are any of polylactic acid and polylactic acid-glycolic acid copolymer or a combination thereof, so that the flexible electrode is biodegradable.

31. The flexible electrode according to claim 1, wherein the implantation portion is provided with a mounting hole through which the electrode implantation device is attached to the flexible electrode for implantation.

32. A manufacturing method of a linear flexible electrode for a peripheral nerve, wherein the flexible electrode is the flexible electrode according to claim 1, comprising:
manufacturing a first insulation layer, a wire layer, a second insulation layer and an electrode site on a substrate; and
separating the flexible electrode from the substrate;
wherein a via hole is manufactured at a position corresponding to the electrode site in at least one layer of the first insulation layer and the second insulation layer by patterning.
